(19) United States
(12) Patent Application Publication
(10) Pub. No.: US 2023/0381169 A1
(43) Pub. Date: Nov. 30, 2023

(54) TREATMENT OF PUBLIC SPEAKING ANXIETY WITH AN ALPHA-7 NICOTINIC ACETYLCHOLINE RECEPTOR AGONIST

(71) Applicant: Vanda Pharmaceuticals Inc., Washington, DC (US)

(72) Inventors: Mihael H. Polymeropoulos, Potomac, MD (US); Christos Polymeropoulos, Potomac, MD (US); Yunsheng He, Fairfax, VA (US)

(21) Appl. No.: 18/249,920

(22) PCT Filed: Nov. 24, 2021
(86) PCT No.: PCT/US2021/060720

§ 371 Date: Apr. 20, 2023

Related U.S. Application Data
(60) Provisional application No. 63/118,121, filed on Nov. 25, 2020.
(60) Provisional application No. 63/118,132, filed on Nov. 25, 2020.
(60) Provisional application No. 63/158,449, filed on Mar. 9, 2021.

(51) Int. Cl. A61K 31/46; A61P 25/18

(52) U.S. Cl. CPC A61K 31/46; A61P 25/18

(57) ABSTRACT

Disclosed herein is a method of treatment or prevention of public speaking anxiety (PSA), which may in some cases present as a symptom of social anxiety disorder (SAD), including the performance only (SAD-PO) subtype of SAD. The method of treatment includes administering an a7 nAChR agonist such as (R)-3-(6-p-tolyl-pyridin-3-yloxy)-1-aza-bicyclo[2.2.2]octane to the individual at a therapeutically effective dose.

### CROSS REFERENCE TO RELATED APPLICATIONS

The present patent application claims priority to U.S. Provisional Patent Application No. 63/118,121, filed Nov. 25, 2020, U.S. Provisional Patent Application No. 63/118,132, filed Nov. 25, 2020, and U.S. Provisional Patent Application No. 63/158,449, filed Mar. 9, 2021.

### BACKGROUND OF THE INVENTION

The invention relates to a method for the treatment of public speaking anxiety (PSA), including public speaking anxiety as a presenting symptom of, or associated with social anxiety disorder (SAD) or social anxiety disorder-performance only subtype (SAD-PO). The invention particularly relates to a method for the treatment of anxiety associated with public speaking or performance as described herein with an alpha 7 nicotinic acetylcholine receptor (α7 nAChR) agonist, particularly with (R)-3-(6-p-tolyl-pyridin-3-yloxy)-1-aza-bicyclo[2.2.2]octane.

Public speaking anxiety (PSA), also known as fear of public speaking or fear of speaking in public, is classified in the American Psychiatric Association's Diagnostic and Statistical Manual of Mental Disorders, 5th edition (DSM-5) (2013) as a social anxiety disorder (SAD). The prevalence of PSA has been reported to be as high as 15% to 30% of the general population, with up to 10% of those with PSA reporting that the condition interferes with daily activities, including work and education. (Vickram Tejwani, M D, et al., Public Speaking Anxiety in Graduate Medical Education—A Matter of Interpersonal and Communication Skills?, Journal of Graduate Medical Education, p. 111 (Feb. 1, 2016).)

Social anxiety disorder (SAD), including PSA, is characterized by excessive fear, discomfort, and self-consciousness in a variety of social situations including e.g., performing in front of others, and giving a speech. SAD is a chronic condition with a typical onset during late childhood and early adolescence, and is often associated with subsequent accrual of comorbid mental health problems such as major depression and substance use disorder. SAD is a multifactorial disease with both environmental and genetic factors as well as their interactions. Generalized SAD is a particularly severe form of SAD, involving all-encompassing fear of social situations. Social anxiety disorder performance only (SAD-PO) is a specifier of SAD based on DSM-5 criteria. This specific type of SAD is typically not associated with comorbid psychiatric conditions or innate behavioral inhibition, and usually has a later onset. SAD-PO arises in narrower circumstances, namely, when a sufferer must perform in public. PSA and symptoms thereof may arise in individuals meeting the diagnostic criteria for SAD, for SAD-PO, for both SAD and SAD-PO, or for neither SAD nor SAD-PO.

The Public Speaking Anxiety Scale (PSAS) is used to assess the three-component model of anxiety. The PSAS consists of seventeen items across three subscales: 1) cognitive (eight items), 2) behavioral (four items), and 3) physiological (five items). Each item has a score ranging from 1 (“not at all”) to 5 (“extremely”), with five items on the scale being reverse coded. The PSAS total score can range from 17 to 85, and is considered a highly reliable and comprehensive measure to assess public speaking anxiety.

Symptoms of PSA, SAD, and SAD-PO may include physiological biomarkers associated with acute stress response, such as elevated heart rate, blood pressure, body temperature, or increased sweating, and other physical, cognitive, and emotional symptoms, e.g., shaking or trembling, blushing, rapid or difficulty breathing, dizziness, nausea, restlessness, muscle tension, sleep disturbances, excessive rumination or worry, difficulty concentrating outside of what is causing the anxiety, a feeling of the sufferer's mind having gone blank, fear of judgment, fear of making a mistake, fear of embarrassment, fear of people looking at the sufferer, fear of certain situations, e.g., ordering at a restaurant or speaking in front of a crowd, engaging in negative self-talk, and other symptoms as known in the art. The emotional, cognitive, and physical symptoms often lead individuals afflicted with such anxiety to alter their behaviors, including refusing invitations, clinging to familiar people in group situations, and avoiding the situations, events, or circumstances that cause them anxiety. Symptoms may frequently manifest in a fear of public speaking or engaging in situations where the individual must be in front of others. Individuals suffering from PSA, SAD, and SAD-PO may further be at risk of developing unhealthy coping mechanisms, including use of drugs and alcohol as a means to relax in anxiety-provoking situations.

Current treatments for SAD and SAD-PO include psychological and pharmacological therapies and their combination. The selective serotonin reuptake inhibitors (SSRIs) paroxetine, sertraline, and fluvoxamine, and the serotonin norepinephrine reuptake inhibitor (SNRI) venlafaxine have been approved by the FDA for SAD in the United States. Other medications including monoamine oxidase inhibitors (MAOIs) and benzodiazepines (BZDs) have showed efficacy in placebo-controlled clinical trials with small sample sizes. The SSRIs increase synaptic serotonin levels and are considered as a first line treatment for SAD, but the onset of action is slow. Additionally, approximately 40% of patients treated with SSRIs report sexual dysfunction associated with SSRI use. BZDs are gamma-aminobutyric acid (GABA)-positive allosteric modulators with adverse effects such as drowsiness, memory disturbances, sedation, and abuse liability. In addition, high rates of partial response and low rates of long-term remission remain.

Many patients with SAD, including SAD-PO, only experience significant distress in events that occur infrequently (e.g. public speaking or social gathering) or only benefit partially from psychological and pharmacological therapies. Therefore, there is an unmet medical need for more effective anxiolytics with improved safety profiles, including those that can be used on an as-needed basis, e.g., for the treatment of public speaking or performance anxiety in patients having or not having a diagnosis of SAD or SAD-PO.

The alpha-7 nicotinic acetylcholine receptor (α7-nAChR) is a ligand-gated ion channel and is involved in many physiologic and pathologic processes. A unique feature of α7-nAChR is high Ca' permeability and fast desensitization. The α7-nAChR is highly expressed in the nervous system, especially in the regions implicated in cognition and memory, and its dysfunction is associated with several neuropsychiatric and neurological disorders including schizophrenia and Alzheimer's disease (AD).

The compound (R)-3-(6-p-tolyl-pyridin-3-yloxy)-1-aza-bicyclo[2.2.2]octane, shown below:

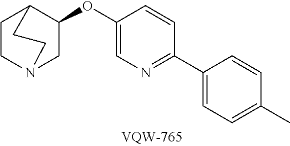 [SMILES: Cc1ccc(-c2ccc(O[C@H]3CN4CCC3CC4)cn2)cc1]

is a potent and selective α7 nAChR agonist disclosed in U.S. Pat. No. 7,579,362. The compound is also known as (R)-3-((6-(p-tolyl)pyridin-3-yl)oxy)quinuclidine, AQW-051, VQW-765, and by the IUPAC name, (3R)-3-{[6-(4-methylphenyl)pyridine-3-yl]oxy}-1-azabicyclo[2.2.2]octane. VQW-765 has a molecular weight of 294.39 g/mol, a pKa (predicted) of 9.09±0.33, and a LogP (predicted) of 3.447±0.402. Pharmaceutically acceptable acid addition salts of (R)-3-(6-p-Tolyl-pyridin-3-yloxy)-1-aza-bicyclo[2.2.2]octane, and particularly the mono-fumarate salt are disclosed in U.S. Pat. No. 9,365,565.

U.S. Pat. No. 7,579,362 describes VQW-765 as an α7 nAChR agonist useful for the prevention and treatment of psychotic disorders such as schizophrenia, mania, depression and anxiety, and for the prevention and treatment of neurodegenerative disorders such as senile dementia, Alzheimer's disease and other intellectual impairment disorders, such as attention deficit hyperactivity disorders (ADHD); Parkinson's disease, Huntington's chorea, amyotrophic lateral sclerosis, multiple sclerosis, epilepsy, convulsions, Tourette syndrome, OCD (obsessive compulsive disorder), neuropathic, postoperative and inflammatory pain, phantom limb pain, cognition, smoking cessation, memory deficits and dysfunction, learning deficit, panic disorders, narcolepsy, nociception, AIDS dementia, senile dementia, autism, tardive dyskinesia, social phobia, pseudodementia.

### BRIEF DESCRIPTION OF THE INVENTION

A first aspect of the disclosure provides a method of treatment of an individual suffering from public speaking anxiety or a symptom thereof, the method comprising: administering to the individual (R)-3-(6-p-tolyl-pyridin-3-yloxy)-1-aza-bicyclo[2.2.2]octane or a pharmaceutically acceptable salt thereof at a dose effective to treat the public speaking anxiety or the symptom thereof. In certain embodiments, the individual may suffer from public speaking anxiety as a presenting symptom of social anxiety disorder (SAD) or SAD-performance only (SAD-PO), and may or may not have been diagnosed with SAD or SAD-PO.

A second aspect of the disclosure provides a method of treating social anxiety disorder (SAD) or a symptom thereof in an individual, the method comprising: administering to the individual (R)-3-(6-p-tolyl-pyridin-3-yloxy)-1-aza-bicyclo[2.2.2]octane or a pharmaceutically acceptable salt thereof at a dose effective to treat the SAD or the symptom thereof. The individual may in certain embodiments suffer from social anxiety disorder-performance only subtype (SAD-PO), and may or may not have been diagnosed with SAD or SAD-PO.

A third aspect of the disclosure provides a method for preventing a manifestation of one or more symptoms of public speaking anxiety in an individual. According to this aspect, the method may comprise administering to the individual a compound prior to engaging in a situation such as, e.g., public speaking or public performance, that is likely to provoke or cause anxiety or one or more symptoms of anxiety. The compound may be (R)-3-(6-p-tolyl-pyridin-3-yloxy)-1-aza-bicyclo[2.2.2]octane or a pharmaceutically acceptable salt thereof, and it may be administered at a dose effective to prevent such a manifestation.

A fourth aspect of the disclosure provides a compound that is (R)-3-(6-p-tolyl-pyridin-3-yloxy)-1-aza-bicyclo[2.2.2]octane or a pharmaceutically acceptable salt thereof, for use in the treatment of public speaking anxiety or a symptom thereof. In various embodiments, the individual may suffer from public speaking anxiety as a presenting symptom of social anxiety disorder (SAD) or SAD-performance only (SAD-PO), and may or may not have been diagnosed with SAD or SAD-PO.

A fifth aspect of the disclosure provides a compound that is (R)-3-(6-p-tolyl-pyridin-3-yloxy)-1-aza-bicyclo[2.2.2]octane or a pharmaceutically acceptable salt thereof, for use in the treatment of social anxiety disorder (SAD) or a symptom thereof. The individual may in certain embodiments suffer from social anxiety disorder-performance only subtype (SAD-PO), and may or may not have been diagnosed with SAD or SAD-PO.

A sixth aspect of the disclosure provides a pharmaceutical composition comprising a compound that is (R)-3-(6-p-tolyl-pyridin-3-yloxy)-1-aza-bicyclo[2.2.2]octane or a pharmaceutically acceptable salt thereof for use in the treatment of public speaking anxiety or a symptom thereof. In various embodiments, the individual may suffer from public speaking anxiety as a presenting symptom of social anxiety disorder (SAD) or SAD-performance only (SAD-PO), and may or may not have been diagnosed with SAD or SAD-PO.

A seventh aspect of the disclosure provides a pharmaceutical composition that is (R)-3-(6-p-tolyl-pyridin-3-yloxy)-1-aza-bicyclo[2.2.2]octane or a pharmaceutically acceptable salt thereof, for use in the treatment of social anxiety disorder or a symptom thereof. The individual may in certain embodiments suffer from social anxiety disorder-performance only subtype (SAD-PO), and may or may not have been diagnosed with SAD or SAD-PO.

An eighth aspect of the disclosure provides a compound that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in the treatment of public speaking anxiety. In various embodiments, the individual may suffer from public speaking anxiety as a presenting symptom of social anxiety disorder (SAD) or SAD-performance only (SAD-PO), and may or may not have been diagnosed with SAD or SAD-PO.

A ninth aspect of the disclosure provides a compound that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in the treatment of social anxiety disorder (SAD). The individual may in certain embodiments suffer from social anxiety disorder-performance only subtype (SAD-PO), and may or may not have been diagnosed with SAD or SAD-PO.

These and other aspects, advantages and salient features of the invention will become apparent from the following detailed description, which describes in greater details various embodiments of the invention.

### DETAILED DESCRIPTION OF THE INVENTION

In various embodiments of the invention, the methods described herein include methods for the treatment of an individual suffering from public speaking anxiety, an individual suffering from social anxiety disorder (SAD) or at least one symptom thereof, or an individual suffering from the performance only subtype of social anxiety disorder (SAD-PO). Such individuals may experience performance or public speaking anxiety as a presenting symptom.

As used herein, references to an individual suffering from social anxiety disorder may be considered to include individuals meeting diagnostic criteria for social anxiety disorder (SAD) as understood by those skilled in the art. For example, such individuals may meet the diagnostic criteria for social anxiety disorder set forth in the American Psychiatric Association's Diagnostic and Statistical Manual of Mental Disorders, 5th edition (DSM-5), pp. 202-208 (2013). Such references are considered to include both individuals diagnosed with SAD, and those who may meet the diagnostic criteria but have not been diagnosed for any number of reasons including, e.g., not having presented for clinical care for SAD or symptoms thereof.

References to an individual suffering from social anxiety disorder-performance only (SAD-PO) may be considered to include individuals meeting diagnostic criteria for the SAD-PO subtype as understood by those skilled in the art. For example, such individuals may meet the diagnostic criteria for the social anxiety disorder-performance only specifier as set forth in the DSM-5. Such references are considered to include both individuals diagnosed with SAD-PO, and those who may meet the diagnostic criteria but have not been diagnosed for any number of reasons including, e.g., not having presented for clinical care for SAD-PO or symptoms thereof.

References to an individual suffering from public speaking anxiety (PSA, also known as fear of public speaking, or fear of speaking in public) may include individuals who experience symptoms of anxiety associated with public speaking or performance. Such anxiety may be referred to as “stage fright,” and may be brought on by any of a number of types of public performance, for example, musical performance, athletic performance, acting performances, and public speaking, or anticipation of such public performance. Such individuals may meet many to all of the diagnostic criteria for SAD, including, e.g., marked anxiety or fear about performing in front of others (e.g., giving a speech). Individuals suffering from PSA may include individuals who are not diagnosed with SAD, but who nonetheless experience PSA. For example, under the DSM-5, an individual who is afraid to speak in public would not receive a diagnosis of social anxiety disorder if this activity is not routinely encountered on the job or in classroom work, and if the individual is not significantly distressed about it. Such individuals may nonetheless be considered to suffer from PSA, and benefit from treatment as described herein. Such individuals may further be identified through objective measures such as, e.g., the Public Speaking Anxiety Scale (PSAS), on which a score of 60 or greater is consistent with identification of an individual as having substantial public speaking anxiety.

As used herein, references to treatment of an individual with one or more of PSA, SAD, or SAD-PO may be considered to include a reduction in severity of the individual's symptoms, prevention or attenuation of progression of PSA, SAD, or SAD-PO in the individual, prevention or attenuation of the individual's symptoms in response to a situation or stimulus, or complete resolution of one or more of the individual's symptoms of PSA, SAD, or SAD-PO after such symptom or symptoms have become manifest in the individual.

In one embodiment, a method is provided for the treatment of an individual suffering from PSA, suffering from SAD, suffering from SAD-PO, or suffering from one or more symptoms of any of the foregoing. Such individual may or may not have been formally diagnosed with any of the foregoing conditions. In particular, this may include an individual suffering from public speaking anxiety or public performance anxiety. In this aspect, the method includes the process of administering to the individual a compound that is (R)-3-(6-p-tolyl-pyridin-3-yloxy)-1-aza-bicyclo[2.2.2]octane or a pharmaceutically acceptable salt thereof in an amount, i.e. at a dose effective to produce the desired therapeutic response. In certain embodiments, the compound may be {2-(R)-3-[[6-(4-Methylphenyl)-3-pyridinyl]oxy]-1-azabicyclo[2.2.2]octane fumarate.

In the methods described herein for treating an individual with PSA, SAD, or SAD-PO, or manifestations or symptoms thereof in such individual, a dose effective to produce the desired therapeutic response is an amount or dosage that is effective to treat the symptoms or underlying PSA, SAD, or SAD-PO in the individual, to shorten the course or lessen the severity of the manifestation of the condition or disorder, or to prevent, mitigate, or ameliorate symptoms of the condition or disorder. The dose for a human may be an amount between about 0.5 mg and about 700 mg as described in

Table 1, e.g., about 0.5 mg, about 2 mg, about 2.5 mg, about 10 mg, about 15 mg, about 75 mg, about 100 mg, about 200 mg, or about 700 mg.

TABLE 1
Daily dose:

| Daily dose | Daily dose for exemplary |
|---|---|
| (mg/kg/day) | 70 kg human (mg) |
| 0.007 | 0.5 |
| 0.03 | 2 |
| 0.04 | 2.5 |
| 0.14 | 10 |
| 0.21 | 15 |
| 1.07 | 75 |
| 1.43 | 100 |
| 2.86 | 200 |
| 10 | 700 |

The dose of the active agent of the present invention may be varied so as to administer an amount of the active agent which is effective to achieve the desired therapeutic response for a particular individual without being toxic to the individual. The selected dosage will depend upon a variety of pharmacokinetic factors including the route of administration, the time of administration, the rate of excretion, the duration of the treatment, other drugs, compounds, or materials used in combination with the particular compositions employed, the age, sex, weight, condition, general health and prior medical history of the individual being treated, and like factors well known in the medical arts.

In another embodiment, a method is provided for treating PSA, SAD, SAD-PO, or at least one symptom thereof in an individual by administering to the individual a compound that is (R)-3-(6-p-tolyl-pyridin-3-yloxy)-1-aza-bicyclo[2.2.2]octane or a pharmaceutically acceptable salt thereof. The compound may be administered at a dosage described herein as one that is therapeutically effective.

In another embodiment, a method of treatment is provided, including administering a compound that is (R)-3-(6-p-tolyl-pyridin-3-yloxy)-1-aza-bicyclo[2.2.2]octane or a pharmaceutically acceptable salt thereof to a human individual suffering from PSA, SAD, or SAD-PO, or symptoms of one of the foregoing. In various embodiments, the individual may or may not have been formally diagnosed with SAD or SAD-PO. In such a method, the compound is administered in a therapeutically effective amount, i.e., an amount sufficient to relieve the at least one symptom of SAD or SAD-PO. Such symptoms of PSA, SAD, and SAD-PO may include physical, cognitive, and emotional symptoms, e.g., shaking or trembling, blushing, increased heart rate, blood pressure, body temperature, or sweating, rapid or difficulty breathing, dizziness, nausea, restlessness, muscle tension, sleep disturbances, excessive rumination or worry, difficulty concentrating outside of what is causing the anxiety, a feeling of the sufferer's mind having gone blank, fear of judgment, fear of making a mistake, fear of embarrassment, fear of people looking at the sufferer, fear of certain situations, e.g., ordering at a restaurant or speaking in front of a crowd, engaging in negative self-talk, and other symptoms as known in the art. The emotional, cognitive, and physical symptoms may also be considered to include behavior alterations including refusing invitations, clinging to familiar people in group situations, and avoiding the situations, events, or circumstances that cause them anxiety, as well as unhealthy coping mechanism such as use of alcohol or drugs.

In a further embodiment, a method is provided for preventing an acute manifestation of one or more symptom of PSA, SAD, or SAD-PO in an individual. Prevention in this context may include prevention in whole or in part, such as a reduction in severity. In such an embodiment, the method includes administering to the individual a compound that is (R)-3-(6-p-tolyl-pyridin-3-yloxy)-1-aza-bicyclo[2.2.2]octane or a pharmaceutically acceptable salt thereof, prior to engaging in a situation likely to provoke anxiety, e.g., prior to participating in a public speaking engagement, and in an amount effective to prevent the symptom(s). In various embodiments, the situation likely to provoke anxiety may be, e.g., performing in front of one or more other people, or public speaking, and the symptom of SAD or particularly of SAD-PO whose manifestation is to be prevented may be, e.g., shaking or trembling, blushing, increased heart rate, rapid or difficulty breathing, dizziness, nausea, restlessness, sweating, muscle tension, sleep disturbances, excessive rumination or worry, difficulty concentrating outside of what is causing the anxiety, a feeling of the sufferer's mind having gone blank, fear of judgment, fear of making a mistake, fear of embarrassment, fear of people looking at the sufferer, fear of certain situations, e.g., ordering at a restaurant or speaking in front of a crowd, and engaging in negative self-talk.

In any of the foregoing methods, the compound may be (R)-3-(6-p-tolyl-pyridin-3-yloxy)-1-aza-bicyclo[2.2.2]octane either in free base form or in a pharmaceutically acceptable acid addition salt form. A pharmaceutically acceptable salt as used herein refers to a salt of a free form that is not toxic, biologically intolerable, or otherwise biologically undesirable. Preferred pharmaceutically acceptable salts are those that are pharmacologically effective and suitable for contact with the tissues of individuals without undue toxicity, irritation, or allergic response. Particularly preferred pharmaceutically acceptable acid addition salts of the present compound are described in U.S. Pat. No. 9,365,565, and include the fumarate, maleate, chloride, phosphate, succinate, or malonate acid addition salt form. In particular, the compound may be (R)-3-(6-p-tolyl-pyridin-3-yloxy)-1-aza-bicyclo[2.2.2]octane in mono-fumarate acid addition salt form, i.e. {2-(R)-3-[[6-(4-Methylphenyl)-3-pyridinyl]oxy]-1-azabicyclo[2.2.2]octane fumarate.

The compound of the present invention can be administered by one or more routes of administration using one or more of a variety of methods known in the art. As will be appreciated by the skilled artisan, the route and mode of administration will vary depending upon the desired results. Routes of administration may include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, a composition can be administered by a nonparenteral route, such as a topical, epidermal or mucosal route of administration, for example, intranasally, orally, vaginally, rectally, sublingually or topically.

The active compounds can be prepared as a solid, immediate release form comprising the compound and one or more pharmaceutically acceptable excipients. Alternatively, the active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

Preferred therapeutic compositions are compositions for oral or transdermal administration. A composition for enteral or parenteral administration is, for example, a unit dosage form, such as a sugar-coated tablet, a tablet, a capsule, a suppository or an ampoule. The unit content of active ingredients in an individual dose need not in itself constitute a therapeutically effective amount, since such an amount can be reached by the administration of a plurality of dosage units. A composition according to the invention may contain, e.g., from about 10% to about 100%, preferably from about 20% to about 60%, of the active ingredients.

If not indicated otherwise, a pharmaceutical composition according to the invention is prepared in a manner known per se, e.g. by means of conventional mixing, granulating, sugar-coating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like. Because of their ease of administration, tablets and capsules represent the most advantageous oral dosage unit forms, in which case solid pharmaceutical carriers are obviously employed.

In particular, the compound may be orally administered in a solid immediate release form, e.g., a tablet or a capsule, comprising VQW-765 in an amount of, e.g., about 0.5 mg, about 2 mg, about 2.5 mg, about 10 mg, about 15 mg, about 75 mg, about 100 mg, about 200 mg, or about 700 mg of the compound, together with one or more pharmaceutically acceptable excipients.

Alternatively, the compound may be orally administered in a solid controlled release form comprising the compound and one or more pharmaceutically acceptable excipients. Controlled release forms may contain larger doses with lower bioavailability as compared to immediate release forms. In further embodiments, the compound may be orally administered in a liquid suspension form, or intravenously administered in a liquid suspension form in a daily dosage providing comparable exposure to the compound.

In another aspect, a compound is provided that is (R)-3-(6-p-tolyl-pyridin-3-yloxy)-1-aza-bicyclo[2.2.2]octane or a pharmaceutically acceptable salt thereof, for use in any of the methods of treatment described herein.

In another aspect, a pharmaceutical composition is provided, the pharmaceutical composition comprising a compound that is (R)-3-(6-p-tolyl-pyridin-3-yloxy)-1-aza-bicyclo[2.2.2]octane or a pharmaceutically acceptable salt thereof for use in any of the methods described herein.

In another aspect, a compound is provided that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in any of the methods described herein.

The skilled artisan will appreciate that additional embodiments may be selected by combining the embodiments above, or by reference to the examples given herein.

### Example 1

### Social Anxiety Disorder Performance Only (SAD-PO); Single Dose

A multicenter, randomized, double-blind, placebo-controlled study is performed to evaluate the efficacy and safety of VQW-765 in patients with substantial public speaking anxiety according to the design in Table 2. In the study, 220 patients are randomized to one of two arms in a 1:1 ratio. Patients in one arm receive capsules containing 10 mg VQW-765 ({2-(R)-3-[[6-(4-Methylphenyl)-3-pyridinyl]oxy]-1-azabicyclo[2.2.2]octane fumarate), while patients in the other arm receive placebo. VQW-765 capsules are size 3, white opaque, hard gelatin capsules provided as a strength of 10 mg, also containing lactose monohydrate, microcrystalline cellulose, methocel, croscarmellose sodium, colloidal silicon dioxide, and magnesium stearate. Placebo capsules are provided in size and appearance identical to those containing VQW-765.

TABLE 2
Clinical trial design

| | Visit 1 | Visit 2 |
|---|---|---|
| Phase | Screening for eligibility | Treatment (Day 1) |
| | (Day −14 to Day −1) | |
| Treatment | | VQW or placebo is given 2 |
| | | hours prior to a TSST |
| | Male and female | Randomization |
| | 18-70 years old | TSST |
| | PSAS ≥ 60 | SUDS, CGI-C, and PGI-C |
| | HAM-D score < 18 | Biomarkers |
| | LSAS | PK samples |
| | Blood samples for | |
| | pharmacogenetics | |

During a screening phase (Table 2, visit 1), the Public Speaking Anxiety Scale (PSAS) is used to determine patients with substantial public speaking anxiety, defined as having a PSAS score greater than or equal to 60. Clinical and laboratory assessments are performed to assess each patient's eligibility. Eligible patients are trained on using a self-rated Subjective Units of Distress Scale (SUDS). In addition, the Liebowitz Social Anxiety Score (LSAS) is administered, and blood samples are collected for pharmacogenetic analysis. LSAS is a clinician-administered rating scale used to assess how social anxiety plays a role in daily life across a variety of situations. The LSAS is used to study outcomes in clinical trials, and includes 24 items to assess both social anxiety in situations and avoidance of those situations. Each item is rated as 0, none; 1, mild; 2, moderate; or 3, severe. The maximal score is 144.

During a treatment phase (Table 2, visit 2), eligible patients are randomized and receive a modified Trier Social Stress Test (TSST) involving a public speaking challenge about 2 hours after administration of study medication. Prior to randomization, patients are asked for a baseline SUDS rating.

Patients are given a single dose of 10 mg VQW-765 or placebo capsule. Two hours later, the patient is instructed to prepare for a 5-minute speech for a job interview, and is asked for a SUDS rating (resting phase). Then the patient has 3 minutes to mentally prepare. SUDS rating is collected at each minute (anticipation phase). Next, the patient gives a 5-minute speech to an audience of two clinical staff dressed in white coats and previously unknown to the patient. SUDS rating is collected just before the speech and then at each minute during the speech (performance phase). The speech is video recorded, and the video camera is observed by the patient. If the patient stops before 5 minutes and keeps silent for about 20 seconds, the lead clinical staff will say “you still have time.” If the patient does not continue, clinical staff will start asking questions until completion of performance phase. Physiological biomarkers associated with anxiety or acute stress response, including heart rate, blood pressure, body temperature, and sweating are monitored continuously by medical devices throughout the Public Speaking challenge.

After the TSST debriefing, the biomarker measurement devices are removed, and Clinician Global Impression-Change (CGI-C) and Patient Global Impression-Change (PGI-C) are administered. Vital signs, safety labs, pharmacokinetic (PK) sample collection and electrocardiogram (ECG) are collected afterward.

The efficacy of a single oral dose of 10 mg VQW-765 is evaluated relative to placebo in change of anxiety rating as measured by: SUDS from baseline to performance phase during a TSST; CGI-C scale; PGI-C scale; and change of biomarkers of anxiety during a TSST. Additionally, safety and tolerability of a single oral dose of 10 mg VQW-765 is assessed, as well as pharmacogenetic assessments to identify genetic factors associated with clinical phenotypes and treatment response. In total, ten SUDS ratings are collected during the TSST: one at introduction to task, three during anticipation phase, and six during the performance phase. Based on a two-sided t-test with the 5% significance level, the planned sample size of 110 patients per arm (a total of 220 patients) provides around 85% power to detect a mean difference of 12 points in the average of SUDS score assuming a standard deviation of 28 in each treatment group.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed, but that the invention will include all embodiments falling within the scope of the appended claims.

What is claimed is:

1. A method of treatment of an individual suffering from public speaking anxiety or a symptom thereof, the method comprising:
administering to the individual a compound that is (R)-3-(6-p-tolyl-pyridin-3-yloxy)-1-aza-bicyclo[2.2.2]octane or a pharmaceutically acceptable salt thereof, at a dose effective to treat the public speaking anxiety or the symptom thereof

2. The method of claim 1, wherein the individual suffers from public speaking anxiety as a presenting symptom of social anxiety disorder (SAD).

3. The method of claim 2, wherein the individual has been diagnosed with SAD.

4. The method of claim 2, wherein the individual suffers from public speaking anxiety as a presenting symptom of SAD-performance only (SAD-PO). The method of claim 4, wherein the individual has been diagnosed with SAD-PO.

6. A method of treatment of an individual suffering from social anxiety disorder (SAD) or a symptom thereof, the method comprising:
administering to the individual a compound that is (R)-3-(6-p-tolyl-pyridin-3-yloxy)-1-aza-bicyclo[2.2.2]octane or a pharmaceutically acceptable salt thereof at a dose effective to treat the SAD.

7. The method of claim 6, wherein the individual has been diagnosed with SAD.

8. The method of claim 6, wherein the individual suffers from SAD-performance only (SAD-PO).

9. The method of claim 8, wherein the individual has been diagnosed with SAD-PO.

10. The method of any one of claims 6-9, wherein the individual suffers from public speaking anxiety as a presenting symptom of the SAD.

11. The method of any one of claims 1-10, wherein the symptom manifests physically in the individual.

12. The method of claim 11, wherein the symptom is selected from the group consisting of: shaking, trembling, blushing, increased heart rate, increased blood pressure, increased body temperature, rapid breathing, difficulty breathing, dizziness, nausea, restlessness, increased sweating, muscle tension, and sleep disturbance.

13. The method of any one of claims 1-10, wherein the symptom manifests cognitively or emotionally in the individual.

14. The method of claim 13, wherein the symptom is selected from the group consisting of: excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.

15. A method of preventing a manifestation of one or more symptoms of public speaking anxiety in an individual, comprising:
prior to participating in a public speaking engagement, administering to said individual a compound that is (R)-3-(6-p-tolyl-pyridin-3-yloxy)-1-aza-bicyclo[2.2.2]octane or a pharmaceutically acceptable salt thereof at a dose effective to prevent said manifestation.

16. The method of claim 15, wherein the one or more symptom is selected from the group consisting of:
shaking, trembling, blushing, increased heart rate, increased blood pressure, increased body temperature, rapid breathing, difficulty breathing, dizziness, nausea, restlessness, increased sweating, muscle tension, sleep disturbances, excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.

17. The method of any one of claims 15-16, wherein the individual suffers from social anxiety disorder (SAD).

18. The method of claim 17, wherein the individual has been diagnosed with SAD.

19. The method of claim 17, wherein the wherein the individual suffers from SAD-performance only (SAD-PO).

20. The method of claim 19, wherein the individual has been diagnosed with SAD-PO.

21. The method of any of claims 1-20, wherein the dose is from about 0.007 mg/kg to about 10 mg/kg.

22. The method of claim 21, wherein the dose is about 0.5 mg, about 2 mg, about 2.5 mg, about 10 mg, about 15 mg, about 75 mg, about 100 mg, about 200 mg, or about 700 mg.

23. The method of any of claims 1-22, wherein the compound is a pharmaceutically acceptable salt of (R)-3-(6-p-tolyl-pyridin-3-yloxy)-1-aza-bicyclo[2.2.2]octane.

24. The method of any of claims 1-23, wherein the compound is (R)-3-(6-p-tolyl-pyridin-3-yloxy)-1-aza-bicyclo[2.2.2]octane in mono-fumarate acid addition salt form.

25. The method of any of claims 1-24, wherein the compound is orally administered in a solid immediate release form comprising the compound and one or more pharmaceutically acceptable excipients.

26. The method of any of claims 1-24, wherein the compound is orally administered in a solid controlled release form comprising the compound and one or more pharmaceutically acceptable excipients.

27. The method of any of claims 1-24, wherein the compound is orally administered in a liquid suspension form.

28. A compound that is (R)-3-(6-p-tolyl-pyridin-3-yloxy)-1-aza-bicyclo[2.2.2]octane or a pharmaceutically acceptable salt thereof, for use in the treatment of public speaking anxiety or a symptom thereof.

29. The compound of claim 28, wherein the individual suffers from public speaking anxiety as a presenting symptom of social anxiety disorder (SAD).

30. The compound of claim 29, wherein the individual has been diagnosed with SAD.

31. The compound of claim 29, wherein the individual suffers from public speaking anxiety as a presenting symptom of SAD-performance only (SAD-PO).

32. The compound of claim 31, wherein the individual has been diagnosed with SAD-PO.

33. A compound that is (R)-3-(6-p-tolyl-pyridin-3-yloxy)-1-aza-bicyclo[2.2.2]octane or a pharmaceutically acceptable salt thereof, for use in the treatment of social anxiety disorder or a symptom thereof.

34. The compound of claim 33, wherein the individual has been diagnosed with SAD.

35. The compound of claim 33, wherein the individual suffers from SAD-performance only (SAD-PO).

36. The compound of claim 35, wherein the individual has been diagnosed with SAD-PO.

37. The compound of any one of claims 33-35, wherein the individual suffers from public speaking anxiety as a presenting symptom of the SAD.

38. The compound of any one of claims 28-37, wherein the symptom manifests physically in the individual.

39. The compound of claim 38, wherein the symptom is selected from the group consisting of: shaking, trembling, blushing, increased heart rate, increased blood pressure, increased body temperature, rapid breathing, difficulty breathing, dizziness, nausea, restlessness, increased sweating, muscle tension, and sleep disturbance.

40. The compound of any one of claims 28-37, wherein the symptom manifests cognitively or emotionally in the individual.

41. The compound of claim 40, wherein the symptom is selected from the group consisting of: excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.

42. A pharmaceutical composition comprising a compound that is (R)-3-(6-p-tolyl-pyridin-3-yloxy)-1-aza-bicyclo[2.2.2]octane or a pharmaceutically acceptable salt thereof for use in the treatment of public speaking anxiety or a symptom thereof.

43. The pharmaceutical composition of claim 42, wherein the individual suffers from public speaking anxiety as a presenting symptom of social anxiety disorder (SAD).

44. The pharmaceutical composition of claim 43, wherein the individual has been diagnosed with SAD.

45. The pharmaceutical composition of claim 43, wherein the individual suffers from public speaking anxiety as a presenting symptom of SAD-performance only (SAD-PO).

46. The pharmaceutical composition of claim 45, wherein the individual has been diagnosed with SAD-PO.

47. A pharmaceutical composition that is (R)-3-(6-p-tolyl-pyridin-3-yloxy)-1-aza-bicyclo[2.2.2]octane or a pharmaceutically acceptable salt thereof, for use in the treatment of social anxiety disorder or a symptom thereof.

48. The pharmaceutical composition of claim 47, wherein the individual has been diagnosed with SAD.

49. The pharmaceutical composition of claim 47, wherein the individual suffers from SAD-performance only (SAD-PO).

50. The pharmaceutical composition of claim 49, wherein the individual has been diagnosed with SAD-PO.

51. The pharmaceutical composition of any one of claims 47-50, wherein the individual suffers from public speaking anxiety as a presenting symptom of the SAD.

52. The pharmaceutical composition of any one of claims 42-51, wherein the symptom manifests physically in the individual.

53. The pharmaceutical composition of claim 52, wherein the symptom is selected from the group consisting of: shaking, trembling, blushing, increased heart rate, increased blood pressure, increased body temperature, rapid breathing, difficulty breathing, dizziness, nausea, restlessness, increased sweating, muscle tension, and sleep disturbance.

54. The pharmaceutical composition of any one of claims 42-51, wherein the symptom manifests cognitively or emotionally in the individual.

55. The pharmaceutical composition of claim 54, wherein the symptom is selected from the group consisting of: excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.

56. A compound that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in the treatment of public speaking anxiety.

57. The compound of claim 56, wherein the individual suffers from public speaking anxiety as a presenting symptom of social anxiety disorder (SAD).

58. The compound of claim 57, wherein the individual has been diagnosed with SAD.

59. The compound of claim 56, wherein the individual suffers from public speaking anxiety as a presenting symptom of SAD-performance only (SAD-PO). The compound of claim 59, wherein the individual has been diagnosed with SAD-PO.

61. A compound that is (R)-3-(6-p-tolyl-pyridin-3-yloxy)-1-aza-bicyclo[2.2.2]octane or a pharmaceutically acceptable salt thereof for use in the manufacture of a pharmaceutical composition comprising the compound for use in the treatment of social anxiety disorder.

62. The compound of claim 61, wherein the individual has been diagnosed with SAD.

63. The compound of claim 61, wherein the individual suffers from SAD-performance only (SAD-PO).

64. The compound of claim 63, wherein the individual has been diagnosed with SAD-PO.

65. The compound of any one of claims 61-64, wherein the individual suffers from public speaking anxiety as a presenting symptom of the SAD.

66. The compound of any one of claims 56-65, wherein the symptom manifests physically in the individual.

67. The compound of claim 66, wherein the symptom is selected from the group consisting of: shaking, trembling, blushing, increased heart rate, increased blood pressure, increased body temperature, rapid breathing, difficulty breathing, dizziness, nausea, restlessness, increased sweating, muscle tension, and sleep disturbance.

68. The compound of any one of claims 56-65, wherein the symptom manifests cognitively or emotionally in the individual.

69. The compound of claim 68, wherein the symptom is selected from the group consisting of: excessive rumination or worry, difficulty concentrating outside of what is causing anxiety, a feeling of the sufferer's mind having gone blank, fear of judgment, fear of making a mistake, fear of embarrassment, fear of people looking at the sufferer, fear of certain situations, and engaging in negative self-talk.
